(19) United States
(12) Patent Application Publication
(10) Pub. No.: US 2020/0185082 A1
(43) Pub. Date: Jun. 11, 2020

(54) MEDICAL INFORMATION PROCESSING SYSTEM AND MEDICAL INFORMATION NOTIFYING METHOD

(71) Applicant: OLYMPUS CORPORATION, Tokyo (JP)

(72) Inventors: Kazuyuki INOKIHARA, Tokyo (JP); Masaru HISANO, Tokyo (JP); Toshiya NISHIMURA, Tokyo (JP)

(73) Assignee: OLYMPUS CORPORATION, Tokyo (JP)

(21) Appl. No.: 16/701,689

(22) Filed: Dec. 3, 2019

Related U.S. Application Data
(63) Continuation of application No. PCT/JP2018/017247, filed on Apr. 27, 2018.

(30) Foreign Application Priority Data
Jun. 12, 2017 (JP) 2017-115458

(51) Int. Cl. G16H 30/20; G16H 40/20; G16H 10/60

(52) U.S. Cl. CPC G16H 30/20; G16H 10/60; G16H 40/20

(57) ABSTRACT

A display request reception unit receives a request for display of an entry screen for entering examination information of a current examination performed on a patient, and a screen generation unit generates an entry screen. A diagnosis detail reception unit receives entry of examination information on the entry screen. An examination information storage unit stores examination information of a past examination. A notification determination unit determines whether or not to give notification regarding a past examination of the same patient after the display request reception unit receives the request for display. A notification execution unit gives predetermined notification regarding the past examination when the notification determination unit determines to give notification.

### BACKGROUND OF THE INVENTION

### 1. Field of the Invention

The present invention relates to a medical information processing system that records examination information for each examination and assists the work of additionally recording examination information for examinations in the past.

### 2. Description of the Related Art

Patent Document 1 discloses an electronic medical record generation system that displays, when the patient is a revisiting patient and the disease name acquired from the past medical record information is a chronic disease, a medical record information entry screen on which the latest medical record information including the same disease name is entered from the past medical record information.
[Patent Document 1] Japanese Patent Application Publication No. 2013-105350

In an endoscopic examination, a doctor inserts an endoscope into a patient's body and displays patient's internal body images on a display device in real time. When the doctor observes an image displayed on the display device and finds an area of concern such as a pathological change or bleeding, the doctor operates a release switch of the endoscope so as to capture a still image of the internal body. After the examination is completed, the doctor enters a diagnosis detail including finding information on a report entry screen, attaches necessary examination images, and creates an examination report.

In endoscopic examinations, accidental symptoms occur with a certain probability in medical practices such as treatments and procedures. According to past surveys, it has been confirmed that accidental symptoms caused due to endoscopic examinations and/or treatments occur in 0.050% of examinations. When a doctor finds an accidental symptom such as perforation or bleeding during an examination, the doctor responds to (treats) the accidental symptoms, and enters and records examination information regarding the accidental symptom on a report entry screen after the examination is completed.

In recent years, the Japan Gastroenterological Endoscopy Society has launched a Japan Endoscopy Database (JED) project and are going forward with the registration, counting, and analyzing of endoscope-related procedure and treatment information throughout Japan. Therefore, a medical information processing system installed in a medical facility needs to be able to accurately record examination information for each examination in order to respond to the JED project.

When a doctor finds the occurrence of an accidental symptom during an examination, the doctor responds to the accidental symptom and enters information regarding the accidental symptom in an examination report. However, an accidental symptom that occurred during the examination may be overlooked, and it may be found later during another examination that the accidental symptom had occurred. At this time, it is necessary to register information regarding the accidental symptom in an examination report of the examination (previous examination) that caused the accidental symptom to occur instead of an examination report of the examination (current examination) where the symptom was found. However, some doctors may mistakenly register the information in the examination report of the current examination. Therefore, it is desired to construct a mechanism for correctly registering information regarding an accidental symptom that occurred during a previous examination in the examination report of the previous examination.

### SUMMARY OF THE INVENTION

In this background, a purpose of the present invention is to provide a technology for assisting the work of entering reports and the like performed by doctors.

A medical information processing system according to one embodiment of the present invention includes: a request reception unit that receives a request for display of an entry screen for entering examination information of a current examination performed on a patient; a screen generation unit that generates an entry screen; an input reception unit that receives entry of examination information on the entry screen; an examination information storage that stores examination information of a past examination; a notification determination unit that determines whether or not to give notification regarding a past examination of the same patient after the request reception unit receives the request for display; and a notification execution unit that gives predetermined notification when the notification determination unit determines to give notification.

Optional combinations of the aforementioned constituting elements and implementations of the invention in the form of methods, apparatuses, systems, recording mediums, and computer programs may also be practiced as additional modes of the present invention.

### BRIEF DESCRIPTION OF THE DRAWINGS

Embodiments will now be described, by way of example only, with reference to the accompanying drawings that are meant to be exemplary, not limiting, and wherein like elements are numbered alike in several figures, in which:

FIG. 1 is a diagram illustrating the configuration of a medical information processing system according to an embodiment;

FIG. 2 is a diagram illustrating an example of a report entry screen;

FIG. 3 is a diagram illustrating an example of a menu window for selecting an image to be attached to a report;

FIG. 4 is a diagram illustrating an example of an examination image selection screen;

FIG. 5 is a diagram illustrating an example of an entry screen for information regarding an accidental symptom;

FIG. 6 is a diagram illustrating an example of notification by a notification execution unit; and

FIG. 7 is a diagram illustrating an example of a postoperative accidental symptom master table.

### DETAILED DESCRIPTION OF THE INVENTION

The invention will now be described by reference to the preferred embodiments. This does not intend to limit the scope of the present invention, but to exemplify the invention.

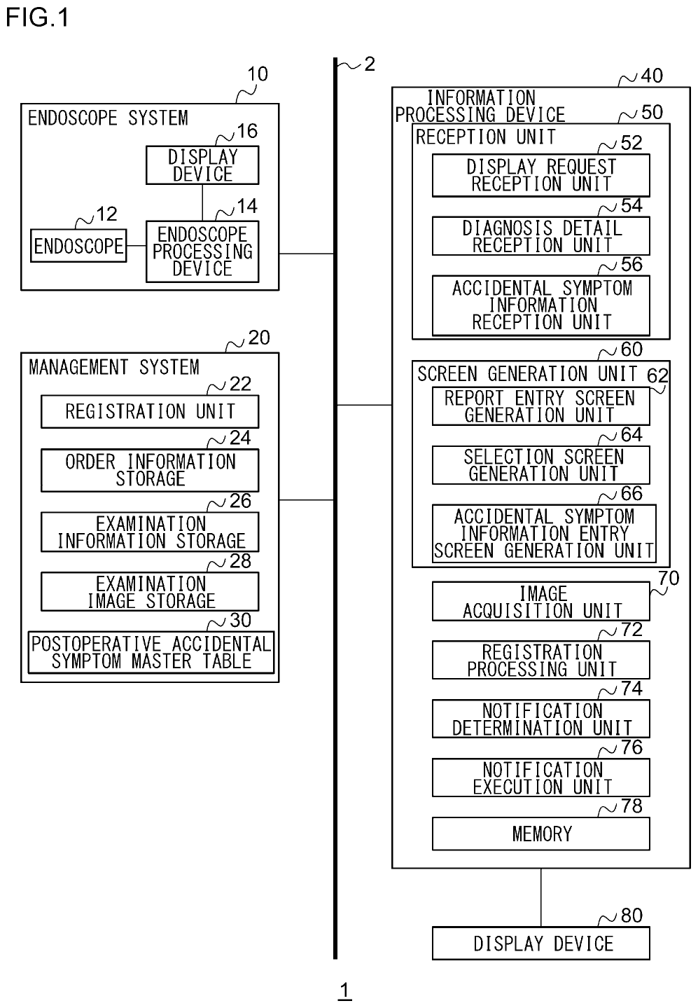

FIG. 1 illustrates the configuration of a medical information processing system 1 according to an embodiment of the present invention. The medical information processing system 1 according to an embodiment is a system for assisting an endoscopic examination report preparation task in a medical facility. The medical information processing system 1 includes an endoscope system 10, a management system 20, and an information processing device 40, which are interconnected by a network 2 such as a LAN (local area network). The information processing device 40 is a terminal device such as a personal computer and is connected to a display device 80 such that output can be displayed on the screen. The information processing device 40 may be a laptop computer integrated with a display device or a portable tablet. Further, the information processing device 40 may be comprised of a combination of a terminal device and a server.

The endoscope system 10 is provided in an endoscopic examination room and includes an endoscope 12, an endoscope processing device 14, and a display device 16. The endoscope 12 is inserted into the patient's body, and a still image of the inside of the body is captured at the time when the doctor presses a release switch of the endoscope 12. The endoscope 12 is provided with a solid-state imaging device (for example, CCD image sensor or CMOS image sensor) and a signal processing circuit. The solid-state imaging device converts incident light into an electrical signal, and the signal processing circuit performs signal processing such as A/D conversion, noise removal, and the like on image data photoelectric-converted by the solid-state imaging device and outputs the resulting image data to the endoscope processing device 14.

The endoscope processing device 14 controls the entire endoscope system 10 in an integrated manner. One important role of the endoscope processing device 14 is to transmit an examination image captured by the endoscope 12 to the management system 20 and store the examination image in an examination image storage 28. Another important role is to display video acquired by the endoscope 12 on the display device 16 in real time.

In the former role, when the release switch of the endoscope 12 is pressed, the endoscope processing device 14 adds information for identifying the examination (examination ID) and image-capturing time information indicating the image-capturing time to an image captured by the endoscope 12 at least as metadata so as to generate examination image data. The endoscope processing device 14 transmits the examination image data to the examination image storage 28 of the management system 20, and the examination image storage 28 stores captured-image data.

The management system 20 includes a registration unit 22, an order information storage 24, an examination information storage 26, an examination image storage 28, and a postoperative accidental symptom master table 30. The order information storage 24 stores order information for an endoscopic examination. For example, examination order information for a patient who has undergone an endoscopic examination within a few days and who complains of pain after the examination sometimes includes information regarding an accidental symptom that may have occurred in a previous examination.

The examination information storage 26 stores examination information of an endoscopic examination and specifically stores examination information entered through the information processing device 40 in association with examination order information such as patient information and examination type information. The examination information includes a diagnosis result such as a diagnosis detail entered by a doctor, a report attachment image selected from among captured examination images, information entered for an accidental symptom, and the like. The information regarding an accidental symptom entered as the examination information includes the type of accidental symptom that occurred and the details of how the accidental symptom was treated.

The examination image storage 28 stores an examination image captured by the endoscope system 10. The examination image storage 28 may be comprised of a large hard disk drive (HDD) or a flash memory.

The information processing device 40 has a function of assisting report preparation performed by a doctor in cooperation with the management system 20. A user interface such as a keyboard and a mouse is connected to the information processing device 40. The information processing device 40 causes the display device 80 to display a screen for an examination image and report preparation, and the doctor operates the user interface while looking at the display screen so as to finish an examination report.

The information processing device 40 accesses the management system 20 so as to display an examination image stored in the examination image storage 28 on the display device 80. When a doctor prepares an endoscopic examination report, thumbnails of all examination images linked to an examination ID and stored in the examination image storage 28 are read out by the information processing device 40, and a thumbnail list is displayed on the display device 80 such that the doctor can select an examination image to be attached to the report.

The information processing device 40 includes a reception unit 50, a screen generation unit 60, an image acquisition unit 70, a registration processing unit 72, a notification determination unit 74, a notification execution unit 76, and a memory 78. The reception unit 50 receives a user interface operation entry from a doctor, for example, a mouse and keyboard operation entry and has a display request reception unit 52, a diagnosis detail reception unit 54, and an accidental symptom information reception unit 56. The screen generation unit 60 generates a screen to be displayed on the display device 80 and includes a report entry screen generation unit 62 that generates a report entry screen for entering examination information, a selection screen generation unit 64 for generating a list screen of examination images captured in an examination, and an accidental symptom information entry screen generation unit 66 that generates an entry screen for entering information regarding the accidental symptom.

The configuration thereof is implemented by hardware such as an arbitrary processor, a memory, or other LSIs and by software such as a program or the like loaded into the memory. The figure depicts functional blocks implemented by the cooperation of hardware and software. Thus, a person skilled in the art should appreciate that there are many ways of accomplishing these functional blocks in various forms in accordance with the components of hardware only, software only, or the combination of both. Further, as described above, although the information processing device 40 may be a terminal device, the information processing device 40 may be formed by a combination of a terminal device and a server. Therefore, each function shown as a component of the information processing device 40 in FIG. 1 may be realized by a device other than a terminal device.

After the completion of an endoscopic examination, the doctor enters a user ID and a password to the information processing device 40 so as to log in. When the doctor logs in, an application for preparing an examination report is automatically started, and a list of already performed examinations for which a report has not been entered yet is displayed on the display device 80. The list of already performed examinations displays examination order information such as the patient name, the patient ID, the examination date and time, the examination type, and the like in a list, and the doctor selects an examination for which a report is to be prepared.

When a doctor selects an examination for which an examination report is to be prepared from a list of examinations that have been performed, the display request reception unit 52 receives a request for display of a report entry screen for entering examination information of the current examination performed on the patient, and the image acquisition unit 70 acquires a thumbnail of an examination image linked to the examination ID of the examination that is selected from the examination image storage 28.

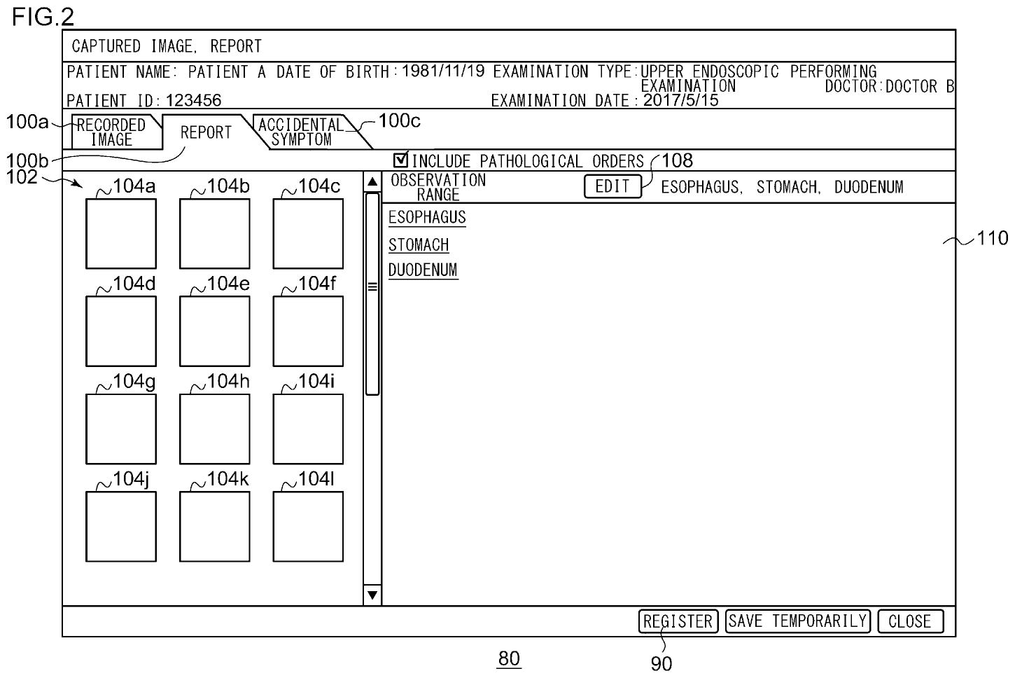

FIG. 2 illustrates an example of report entry screen. When the display request reception unit 52 receives a request for display of a report entry screen for entering the examination information of the current examination, the report entry screen generation unit 62 generates a report entry screen and causes the display device 80 to display the report entry screen. During the display of the report entry screen, a report tab 100b is being selected. In the upper part of the report entry screen, information on a patient name, a patient ID, the date of birth, an examination type, an examination date, and a performing doctor obtained from the order information storage 24 is displayed. The report entry screen is composed of two areas. An examination image display area 102 for displaying thumbnails of examination images is provided on the left side, and an examination result entry area 110 for a doctor to enter examination results such as diagnosis details is provided on the right side.

The report entry screen generation unit 62 displays thumbnails 104a to 1041 of the examination images acquired by the image acquisition unit 70 from the examination image storage 28 side by side in the examination image display area 102. A scroll bar is provided on the right side of the examination image display area 102, and the doctor can scroll the scroll bar to observe all the thumbnails of the examination images. The doctor selects an examination image to be attached to a report while observing the examination images.

The examination result entry area 110 is an area for a doctor to enter an examination result. In the example shown in the figure, an area is provided for entering diagnosis details for “esophagus”, “stomach”, and “duodenum” that are observation ranges in an upper endoscopic examination are displayed. When an edit button 108 is operated, selections for an observation range are displayed in a menu window so that the doctor can select an observation range to be diagnosed. The examination result entry area 110 may have a format where a plurality of selections are displayed for examination information such that the doctor enters a diagnosis detail by selecting a check box or may have a free format for free text entry.

In the report entry screen, the doctor selects an examination image to be attached to the report from the examination image display area 102 and enters the examination result including a diagnosis detail in the examination result entry area 110. The diagnosis detail reception unit 54 receives examination information including the diagnosis detail entered by the doctor on the report entry screen.

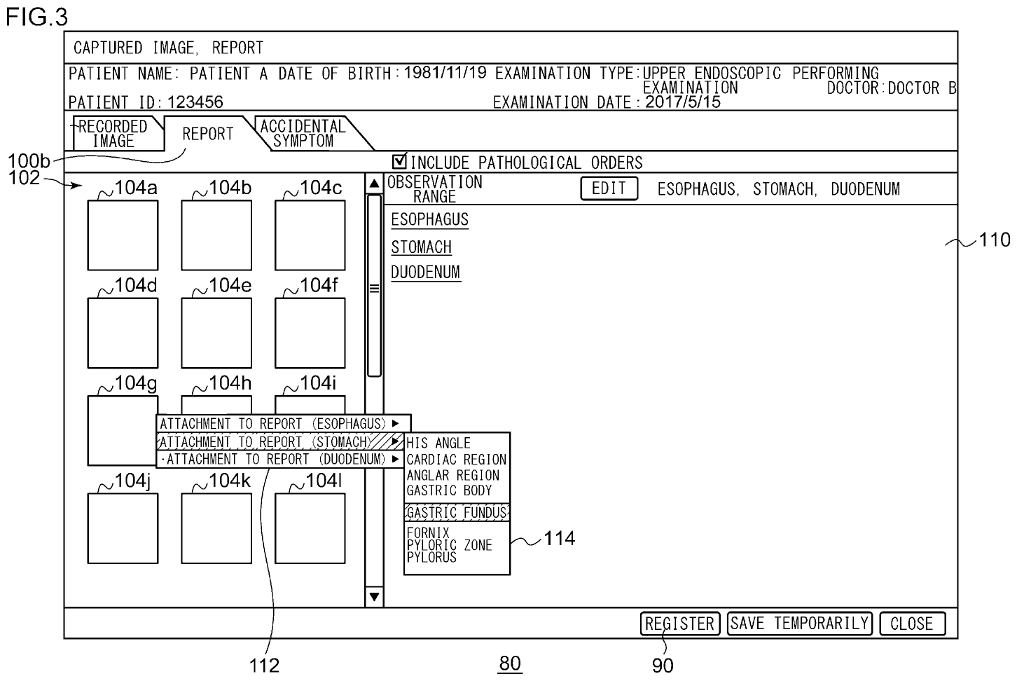

FIG. 3 illustrates an example of a menu window for selecting an image to be attached to a report in a report entry screen. When the doctor selects a thumbnail 104 to be attached to the report in the examination image display area 102 and right-clicks the mouse, a part selection window 112 is displayed. The part selection window 112 displays an observation range for attaching an image. Three selections in the part selection window 112 are, in order from the top, an item for the attachment to the diagnosis result for the observation range “esophagus”, an item for the attachment to the diagnosis result for “stomach”, and an item for the attachment to the diagnosis result for “duodenum”.

When the doctor selects any of the items using a mouse pointer in the part selection window 112, a detailed part window 114 is displayed in which detailed parts included in the observation range are listed. The detailed part window 114 displays the detailed parts included in the observation range, and the doctor selects any of the detailed parts using the mouse pointer. In this example, as the detailed parts of the stomach, items “his angle”, “cardiac region”, “angular region”, “gastric body”, “gastric fundus”, “fornix”, “pyloric zone”, and “pylorus” are set. FIG. 3 shows a situation where the doctor selects a thumbnail 104h, “attachment to report (stomach)” in the part selection window 112, and “gastric fundus” in the detailed part window 114.

The diagnosis detail reception unit 54 receives an operation for selecting an examination image to be attached to an endoscopic examination report. When the diagnosis detail reception unit 54 receives an examination image selection operation, the memory 78 stores that the examination image of the thumbnail 104h is set as an attachment image of the diagnosis result of the stomach and that this image is an image obtained by capturing the image of the “gastric fundus” of the “stomach”. As described above, the doctor selects an examination image to be attached to a report.

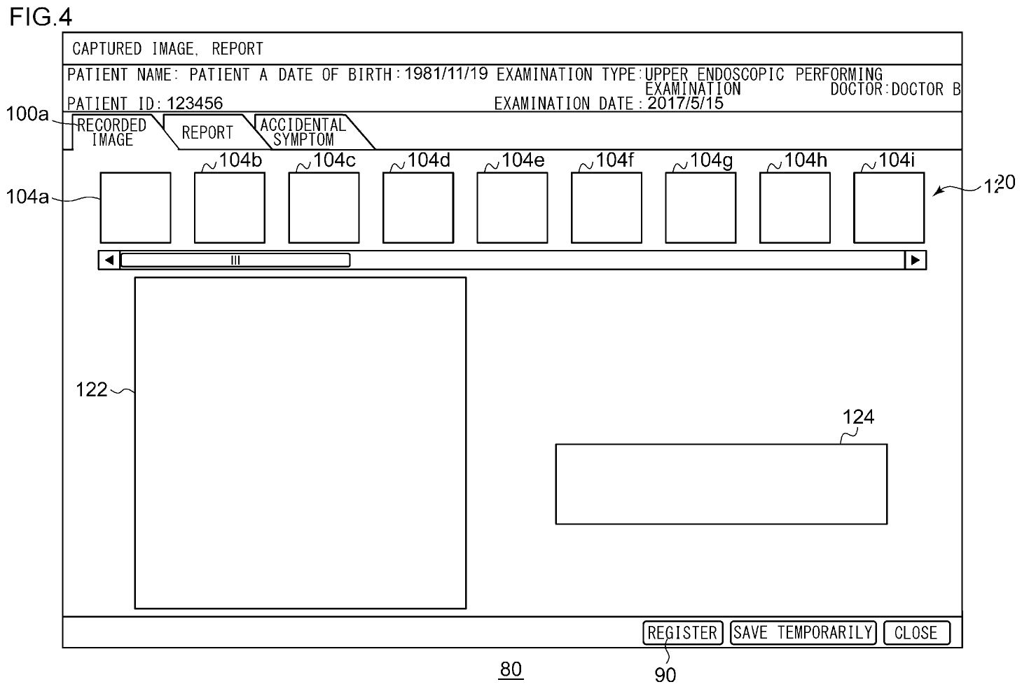

FIG. 4 illustrates an example of an examination image selection screen. The selection screen generation unit 64 generates an examination image selection screen. During the display of the selection screen, the recorded image tab 100a is being selected. In the upper part of the selection screen, information on a patient name, a patient ID, the date of birth, an examination type, an examination date, and a performing doctor obtained from the order information storage 24 is displayed. Below the order information, an examination image display area 120 for displaying thumbnails of examination images is provided. The selection screen generation unit 64 displays thumbnails of the examination images acquired by the image acquisition unit 70 from the examination image storage 28 side by side in the examination image display area 120. A scroll bar is provided below the examination image display area 120, and the doctor can scroll the scroll bar to observe all the thumbnails of the examination images.

The doctor can select a report attachment image from either the report entry screen or the selection screen. Also on the selection screen, the part selection window 112 and the detailed part window 114 shown in FIG. 3 are displayed according to the mouse operation by the doctor, and the doctor can register a report attachment image in the examination information storage 26 along with the examination result such as diagnosis detail. An advantage of the selection screen shown in FIG. 4 is that an enlarged display area 122 for displaying the thumbnail 104 in an enlarged manner is provided. When the doctor selects the thumbnail 104 in the examination image display area 120, the thumbnail 104 that is selected is displayed in an enlarged manner in the enlarged display area 122.

The doctor can accurately observe the presence of a lesion from an examination image displayed in an enlarged manner and can effectively determine whether or not to attach the examination image to a report. The doctor can enter a comment regarding the image displayed in an enlarged manner in the enlarged display area 122 in a comment entry field 124. For example, when a display image is reddish, a comment stating “bleeding is confirmed” may be entered in the comment entry field 124. When the bleeding is caused by a treatment or the like in an examination in the past, a comment stating “bleeding caused by the treatment in the previous examination is confirmed” may be entered in the comment entry field 124. The comment entered in the comment entry field 124 is stored in the memory 78 in association with the image. Operating the registration button 90 later causes the comment stored in the memory 78 to be stored in the examination information storage 26 as the examination result along with the diagnosis detail, the report attachment image, etc.

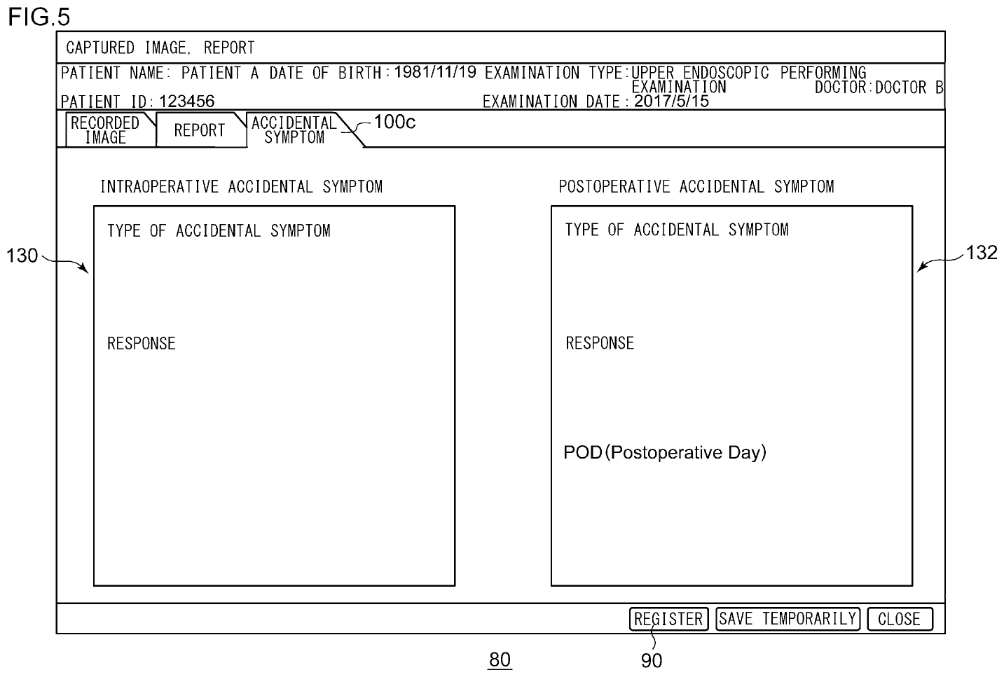

FIG. 5 illustrates an example of an entry screen for information regarding an accidental symptom. When an accidental symptom tab 100c is selected, the accidental symptom information entry screen generation unit 66 generates an entry screen for information regarding an accidental symptom. In the upper part of an accidental symptom information entry screen, information on a patient name, a patient ID, the date of birth, an examination type, an examination date, and a performing doctor obtained from the order information storage 24 is displayed. Below the order information, an intraoperative accidental symptom entry area 130 for entering information regarding an intraoperative accidental symptom and a postoperative accidental symptom entry area 132 for entering information regarding a postoperative accidental symptom are provided.

The intraoperative accidental symptom entry area 130 is an area for entering information regarding an accidental symptom that has occurred and has been treated during an examination. An accidental symptom found during an examination is called “intraoperative accidental symptom”. As information regarding an accidental symptom, the doctor enters the type of the accidental symptom and the treatment to the accidental symptom, and the accidental symptom information reception unit 56 receives the entry. The types of accidental symptoms include perforation, bleeding, etc. The intraoperative accidental symptom entry area 130 may have a format where a plurality of selections are displayed for the type of the accidental symptom and the treatment such that the doctor enters the type and the treatment by selecting a check box or may have a free format for free text entry.

When the doctor operates a registration button 90 after entering the examination information, the registration processing unit 72 transmits information stored in the memory 78 to the management system 20. In the memory 78, in addition to a diagnosis result entered on the report entry screen, an examination image selected as a report attachment image, a comment entered for the examination image, and the examination information including information regarding an intraoperative accidental symptom entered in the intraoperative accidental symptom entry area 130, order information such as the patient name, the patient ID, the date of birth, the examination type, the examination date, and the performing doctor is stored. The registration processing unit 72 instructs the management system 20 to register the examination information and the order information in the examination information storage 26.

In the management system 20, based on an instruction from the registration processing unit 72, the registration unit 22 stores the examination information in the examination information storage 26 as the report information in association with the doctor ID for identifying the doctor who has entered the examination information. The report information stored in the examination information storage 26 can be printed in a predetermined format and used as an examination report.

Gastrointestinal perforation in an examination for making a diagnosis is often caused due to mucosal damage by an endoscope. Usually, a strong abdominal pain appears when perforation occurs. When the doctor recognizes that perforation has occurred during an examination, the doctor responds to the perforation by reefing a perforated site with, for example, a clip. However, if the perforation is large, an emergency surgical operation may be required.

Gastrointestinal perforation caused due to treatment may occur during the treatment or may occur after a certain period of time after the treatment. Perforation that occurs after a certain period of time is considered to be caused by the falling off of a weakened tissue due to the heat generated by an endoscope treatment instrument. Therefore, even if no perforation is found during the examination, a situation may occur in which the patient complains of abdominal pain and undergoes a reexamination after the examination. As described above, when a doctor responds to an accidental symptom occurred during an examination, information regarding the accidental symptom is entered to the intraoperative accidental symptom entry area 130 at the time of entering the examination information.

On the other hand, there are cases where perforation occurs after the examination, and cases where bleeding occurs during the examination but the doctor finishes the endoscopic examination without noticing the bleeding. At this time, the doctor cannot enter information regarding the accidental symptom in the examination report because the accidental symptom has not occurred yet or the doctor is not aware of the occurrence of the accidental symptom although the examination is the factor of the accidental symptom.

In such cases, when the patient complains of a bad physical condition after the examination is completed and undergoes an endoscopic examination again, it turns out that an accidental symptom such as perforation and/or bleeding has occurred due to the previous examination. Such an accidental symptom found after the examination is completed is called “postoperative accidental symptom”. Since the postoperative accidental symptom found in the current examination is caused due to the previous examination, the postoperative accidental symptom needs to be recorded as examination information for the previous examination in which the postoperative accidental symptom occurred instead of being recorded as examination information for the current examination.

The postoperative accidental symptom entry area 132 in FIG. 5 is an area for entering information regarding an accidental symptom found after an examination. Therefore, the postoperative accidental symptom entry area 132 is not used for entering examination information for the current examination. Instead, examination information already registered in the examination information storage 26 is displayed on the display device 80 again, and the postoperative accidental symptom entry area 132 is used for entering information regarding a postoperative accidental symptom so as to add the information to the examination information of the past examination (previous examination).

When it is assumed that information regarding accidental symptoms is collected and statistically processed, it is very important to record the information regarding accidental symptoms while correctly linking the information to respective examinations that caused the accidental symptoms. However, sometimes the doctor erroneously enters information regarding an accidental symptom occurred in the previous examination as examination information for the current examination, that is, the doctor erroneously enters the information in an intraoperative accidental symptom entry area 130 (or postoperative accidental symptom entry area 132) at the time of preparing a report for the current examination. Accordingly, in the medical information processing system 1 according to the embodiment, for the purpose of recording information regarding an accidental symptom in association with the correct examination, a mechanism is provided that gives predetermined notification regarding an examination in the past to the doctor trying to prepare a report for the current examination.

First, as a presupposition, the examination information storage 26 stores examination information of examinations in the past. When preparing a report for the current examination, the display request reception unit 52 receives a request for display of a report entry screen, and the notification determination unit 74 determines whether or not to give notification regarding a past examination of the same patient as that for the current examination at a timing after the display request reception unit 52 receives the request for display. The timing after the display request reception unit 52 receives the request for display may be immediately after receiving the request for display, may be the timing when the doctor makes an entry on the entry screen for the current examination, or may be the timing when the report preparation for the current examination is completed. The notification execution unit 76 gives predetermined notification regarding the past examination when the notification determination unit 74 determines to give notification.

The notification determination unit 74 determines to give notification regarding the past examination when there is a possibility that examination information exists that is to be added to the examination information of the past examination of the same patient. That is, the notification determination unit 74 determines to give notification regarding the past examination when it is determined that there is a possibility that a postoperative accidental symptom has been found in the current examination. Hereinafter, the mode of notification made by the notification execution unit 76 will be described along with notification timing variations.

(Notification Mode 1)

The notification determination unit 74 determines to give notification regarding the past examination when information regarding an accidental symptom is included in order information of an examination performed on the patient. For example, if a patient who has undergone an endoscopic examination within a few days complains of a bad physical condition, the doctor may register an examination reason indicating that an accidental symptom is suspected in the order information when preparing an examination order. For example, when EMR (endoscopic mucosal resection) was performed in the previous examination, the doctor enters “bleeding after EMR suspected” as the examination reason for the current examination. After the display request reception unit 52 receives a request for display of a report entry screen for entering the examination information for the current examination, the notification determination unit 74 refers to the examination reason included in the order information of the examination and determines whether or not the examination reason includes information regarding a postoperative accidental symptom. At this time, if information regarding a postoperative accidental symptom is included in the examination reason, the notification determination unit 74 determines that there is a possibility that the postoperative accidental symptom has been found in the current examination and determines to give notification regarding the past examination.

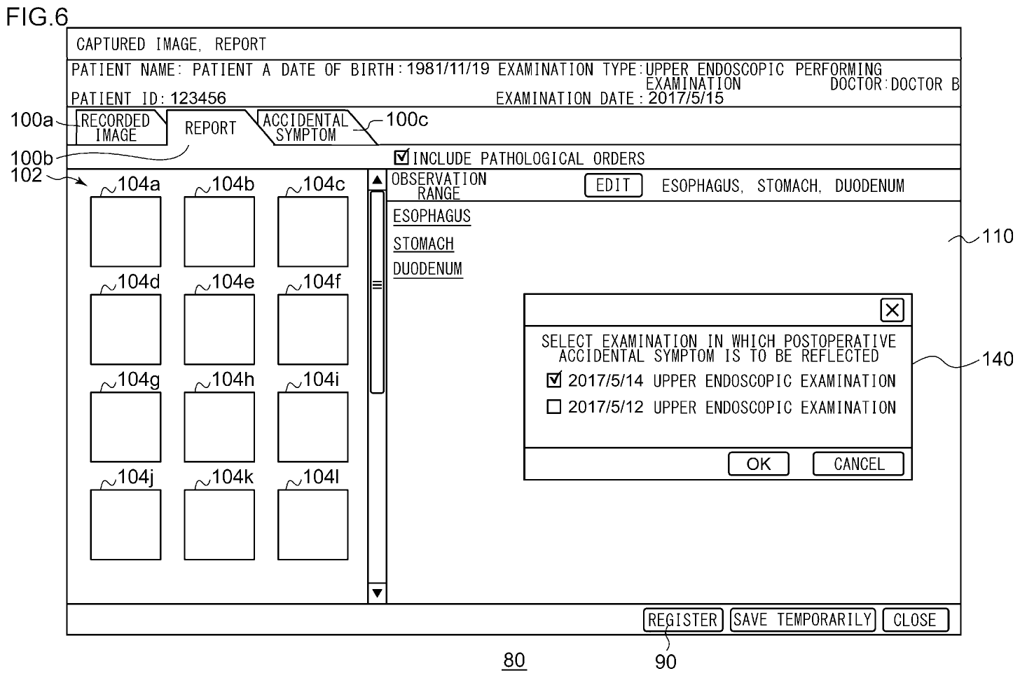

FIG. 6 illustrates an example of notification by the notification execution unit 76. When the notification determination unit 74 determines to give notification regarding the past examination of the same patient, the notification execution unit 76 generates a notification screen 140 for giving notification of an examination performed before the current examination as information regarding the past examination and displays the notification screen 140 on the display device 80.

On the notification screen 140, information regarding examinations performed on the same patient A before the current examination is displayed in a selectable manner. When the doctor finds an accidental symptom that has occurred in a past examination during the current examination, the doctor selects the past examination as notified on the notification screen 140. On the notification screen 140, information (examination date and examination type) specifying the past examinations on the same patient is displayed in a list, and check boxes for selecting the past examinations are provided. By selecting a check box, the doctor can select a past examination that caused the accidental symptom. On the notification screen 140 shown in FIG. 6, an upper endoscopic examination whose examination date is 2017 May 14 is being selected.

In the embodiment, the current examination performed on the patient A is performed by a doctor B. If the upper endoscopic examination selected on the notification screen 140 was also performed by the doctor B, the report entry screen generation unit 62 reads examination information of the upper endoscopic examination of the patient A whose examination date is 2017 May 14 from the examination information storage 26 and displays the report entry screen to which the read examination information is entered on the display device 80. The doctor B selects an accidental symptom tab 100c and enters information regarding a postoperative accidental symptom in the postoperative accidental symptom entry area 132.

As described, when there is a possibility that examination information exists that is to be added to the examination information of the past examination of the same patient A, the notification determination unit 74 determines to give notification regarding the past examination, and the notification execution unit 76 gives notification regarding the past examination. Thereby, the doctor B can easily learn the past examination to which information regarding the postoperative accidental symptom is to be entered. In particular, in the embodiment, since examination information of a past examination is automatically read out and displayed on the report entry screen when the doctor B simply selects the past examination on the notification screen 140, information regarding a postoperative accidental symptom can be easily entered in the postoperative accidental symptom entry area 132. As described, notification of information regarding a past examination given by the notification execution unit 76 allows the doctor to additionally record information regarding a postoperative accidental symptom appropriately as examination information of the past examination.

If the upper endoscopic examination selected on the notification screen 140 was performed by a doctor other than the doctor B, information regarding a postoperative accidental symptom may be preferably recorded by the doctor who performed a past examination that caused the accidental symptom. Therefore, if the past examination was performed by a different doctor, the notification execution unit 76 notifies the different doctor to enter the information regarding the postoperative accidental symptom so as to add the information to the examination information of the upper endoscopic examination of the patient A whose examination date is 2017 May 14. For example, in the medical information processing system 1, when a mechanism for notifying each doctor of a work list is constructed, the notification execution unit 76 may include the additional entry of information regarding a postoperative accidental symptom in the corresponding doctor's work list.

As described above, when the order information of the current examination includes information regarding an accidental symptom, the notification determination unit 74 determines that there is a possibility that information regarding an accidental symptom exists that should be added to the examination information of a past examination of the same patient. Accordingly, the notification determination unit 74 determines to give notification regarding the past examination, and the notification execution unit 76 gives notification of information regarding the past examination.

However, when preparing order information for the current examination, the doctor may not register an examination reason indicating that an accidental symptom is suspected. Therefore, the notification determination unit 74 is preferably able to determine to give notification regarding a past examination to which postoperative accidental symptom information should be added, based also on information other than the order information.

(Notification Mode 2)

As shown in FIG. 3, the doctor B who is the performing doctor of the current examination enters examination information including a diagnosis detail in the examination result entry area 110 while selecting an examination image to be attached to the report on the report entry screen. When the doctor performs a treatment, the treatment performed in the examination and an instrument used in the treatment are also entered in the examination result entry area 110. In the current examination, if the doctor finds bleeding occurring as a postoperative accidental symptom, the doctor performs treatment such as application of a hemostatic agent such as thrombin and endoscopic clipping. At the time of report entry, the doctor enters “hemostasis” as the treatment and “hemostatic agent” or “clip” as the instrument in the examination result entry area 110.

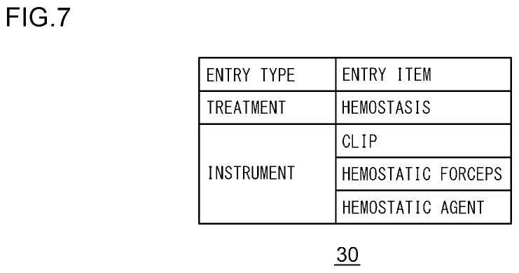

FIG. 7 illustrates an example of the postoperative accidental symptom master table 30. The postoperative accidental symptom master table 30 defines an entry type and an entry item that indicate the possibility of having performed treatment for a postoperative accidental symptom in the current examination. On the report entry screen, the diagnosis detail reception unit 54 receives examination information including the diagnosis detail entered by the doctor on the report entry screen. For example, when “hemostasis” is entered as “treatment” or when any one of “clip”, “hemostatic forceps”, and “hemostatic agent” is entered as “instrument”, the notification determination unit 74 determines that there is a possibility that a postoperative accidental symptom has been treated.

However, although there is a possibility that the postoperative accidental symptom has been treated, it is a common practice to stop bleeding or use an instrument such as a clip for stopping bleeding during an examination. Thus, when the diagnosis detail reception unit 54 receives the entry of examination information recorded in the postoperative accidental symptom master table 30, the notification determination unit 74 according to the embodiment determines whether or not the postoperative accidental symptom has been treated during the current examination using a temporal condition.

When a postoperative accidental symptom occurs, the patient often undergoes an endoscopic examination within a few days since the pain does not go away after the completion of the examination. To put it differently, a case where a re-examination is performed several days after the previous examination can be considered to be a re-examination that does not result from a postoperative accidental symptom. Thus, when the diagnosis detail reception unit 54 receives the entry of examination information recorded in the postoperative accidental symptom master table 30, on condition that examination information of a past examination performed within a predetermined number of days from the date the current examination is performed is stored in the examination information storage 26, the notification determination unit 74 may determine to give notification regarding the past examination. For example, the predetermined number of days may be “three days”. In this case, if the patient underwent an endoscopic examination within three days from the current examination and the bleeding was stopped or an instrument for stopping bleeding was used during the current examination, the notification determination unit 74 determines that there is a possibility that a postoperative accidental symptom was treated during the current examination and determines to give notification regarding the past examination. Thereby, the notification execution unit 76 notifies the doctor of information regarding the past examination. This notification may be performed through the notification screen 140 shown in FIG. 6.

The notification determination unit 74 may determine to give notification regarding the past examination on condition that the examination information of the past examination performed within the predetermined number of days from the date the current examination is performed includes the same examination type information as that of the current examination. For example, even when the patient underwent an endoscopic examination within three days from the date the current examination is performed, there is no possibility that a postoperative accidental symptom occurred in the previous examination if the current examination is an upper endoscopic examination while the previous examination is a lower endoscopic examination. Therefore, the notification determination unit 74 preferably determines whether or not to give notification regarding a past examination on condition that the examination type of the past examination is the same as the examination type of the current examination.

On condition that treatment or the like was performed in the previous examination, the notification determination unit 74 may determine to give notification regarding the past examination. The treatment or the like includes EMR, ESD, polypectomy, and the like. It is known that the probability of the occurrence of an accidental symptom is higher in a case where treatment was performed during an examination compared to a case where treatment was not performed. Accordingly, on condition that the examination information of a past examination performed within the predetermined number of days from the date the current examination is performed includes the same examination type information as that of the current examination and that treatment or the like was performed in the past examination, the notification determination unit 74 may determine to give notification regarding the past examination.

In the notification mode 2, the condition for the number of days and the entry items defined in the postoperative accidental symptom master table 30 may be able to be changed appropriately in a medical facility. In other words, depending on the medical facility, the number of days used as the search range for past examinations may be two days, and more entry items than those shown in FIG. 7 may be registered in the postoperative accidental symptom master table 30. For example, although FIG. 6 displays a list of past examinations performed within three days from the current examination date, a past examination performed on 2017 May 12 will not be displayed on the notification screen 140 if the predetermined number of days is set to “two days”.

(Notification Mode 3)

In the notification mode 2, the notification determination unit 74 determines to give notification regarding a past examination on condition that an entry item defined in the postoperative accidental symptom master table 30 is entered on the report entry screen. In a notification mode 3, an explanation will be given with regard to determination as to whether or not to give notification regarding a past examination when the doctor enters information regarding an accidental symptom shown in FIG. 5 on an entry screen.

First, when entering the examination information of the current examination, there is basically no chance for a doctor to enter information regarding an accidental symptom in the postoperative accidental symptom entry area 132. This is because the postoperative accidental symptom entry area 132 is an entry field used when registering information regarding a postoperative accidental symptom so as to add the information to examination information of a past examination for which the examination information has already been entered and is not to be used as an entry field for examination information of the current examination. Therefore, for the entry of the examination information of the current examination, when the accidental symptom information reception unit 56 receives an entry in postoperative accidental symptom entry area 132, the notification determination unit 74 determines to give notification regarding a past examination, and the notification execution unit 76 gives predetermined notification regarding the past examination. As described in the notification mode 2, a past examination subjected to notification may be on condition that the examination was performed within a predetermined number of days from the current examination date and/or has the same examination type as that of the current examination.

First, it is assumed that, when entering the examination information of the current examination, a case where the doctor enters information regarding an accidental symptom in the postoperative accidental symptom entry area 132 occurs due to the doctor not correctly understanding that the information should be entered in a postoperative accidental symptom entry area 132 for a past examination. Thus, presenting a notification screen 140 (see FIG. 6) by the notification execution unit 76 allows the doctor to be aware of his/her misunderstanding and to record the information regarding the postoperative accidental symptom so as to add the information to the examination information of the past examination.

Also, when entering the examination information of the current examination, there is basically no problem for a doctor to enter information regarding an accidental symptom in the intraoperative accidental symptom entry area 130. This is because in a case where an intraoperative accidental symptom has occurred indeed, information regarding the intraoperative accidental symptom must be entered in the intraoperative accidental symptom entry area 130. However, there still remains a possibility that the doctor mistakenly enters information regarding a postoperative accidental symptom from a past examination in the intraoperative accidental symptom entry area 130 for the current examination.

Therefore, when the condition explained in the notification mode 2, that is, the condition where a past examination of the same examination type was performed within a predetermined number of days from the current examination is satisfied, the notification determination unit 74 may determine to give notification regarding the past examination. At this time, the notification execution unit 76 displays a notification screen 140 over the entry screen shown in FIG. 5. If there is no problem with the entry in an intraoperative accidental symptom entry area 130, the doctor just needs to operate a cancel button on the notification screen 140 so as to hide the notification screen 140. If the doctor sees the notification screen 140 and realizes that the information should be entered as information regarding a postoperative accidental symptom in a past examination, the doctor deletes the information entered in the intraoperative accidental symptom entry area 130 for the current examination and registers the information regarding the postoperative accidental symptom so as to add the information to the examination information of the past examination.

Described above is an explanation based on the embodiment of the present invention. The embodiment is intended to be illustrative only, and it will be obvious to those skilled in the art that various modifications to constituting elements and processes could be developed and that such modifications are also within the scope of the present invention.

In the embodiment, the conditions for determining to give notification regarding a past examination are shown in the notification modes 1 to 3. The conditions shown in the respective notification modes may be used not only in the respective notification modes but also in other notification modes. In other words, in an exemplary variation, the conditions shown in the notification modes 1 to 3 may be combined in various ways so as to determine to give notification regarding a past examination.

In the embodiment, an example of an entry screen for information regarding an accidental symptom is shown in FIG. 5. However, an accidental symptom information entry screen is not limited to this. An accidental symptom information entry screen may include not only an intraoperative accidental symptom entry area 130 for entering information regarding an intraoperative accidental symptom and a postoperative accidental symptom entry area 132 for entering information regarding a postoperative accidental symptom but also an area for entering different type of information. For example, the accidental symptom information entry screen may include an area for entering the degree of atrophy according to the Kimura-Takemoto classification and may also include an area for entering other information. Further, in the embodiment, an accidental symptom information entry screen is displayed upon the selection of an accidental symptom tab 100c. Alternatively, the accident information entry screen may be constructed such that the accident information entry screen is displayed through a different user interface such as a link button instead of being displayed through the selection of a tab.

What is claimed is:

1. A medical information processing system, comprising:
a processor comprising hardware; and
an examination information storage that stores examination information of a past examination,
wherein the processor is configured to:
receive a request for display of an entry screen for entering examination information of a current examination performed on a patient;
determine, when there is a possibility that examination information exists that is to be added to examination information of the past examination of the same patient after receiving the request for display, to give notification regarding the past examination; and
give predetermined notification.

2. The medical information processing system according to claim 1,
wherein the processor is configured to:
receive entry of examination information on the entry screen.

3. The medical information processing system according to claim 1,
wherein the processor is configured to:
determine to give notification regarding a past examination when information regarding an accidental symptom is included in order information of an examination performed on a patient.

4. The medical information processing system according to claim 2,
wherein the processor is configured to:
determine, if examination information of a past examination performed within a predetermined number of days from the date the current examination is performed is stored in the examination information storage, to give notification regarding the past examination when receiving entry of predetermined examination information.

5. The medical information processing system according to claim 4,
wherein the processor is configured to:
determine to give notification regarding the past examination if the examination information of the past examination performed within the predetermined number of days from the date the current examination is performed includes the same examination type information as that of the current examination.

6. The medical information processing system according to claim 4,
wherein the predetermined examination information is information that indicates that hemostatic treatment has been performed.

7. The medical information processing system according to claim 6,
wherein the information that indicates that hemostatic treatment has been performed is information that indicates an instrument used to stop bleeding.

8. The medical information processing system according to claim 2,
wherein the processor is configured to:
determine to give notification regarding the past examination when receiving information entry on an entry screen for information regarding an accidental symptom.

9. The medical information processing system according to claim 1, comprising:
an endoscope system that has an endoscope and a processing device;
a management system that manages examination information of an endoscopic examination; and
an information processing device that is operated by a doctor.

10. A medical information notifying method comprising:
receiving a request for display of an entry screen for entering examination information of a current examination performed on a patient;
determining, when there is a possibility that examination information exists that is to be added to examination information of a past examination of the same patient after receiving the request for display, to give notification regarding the past examination; and
giving predetermined notification.
